(19) United States
(12) Patent Application Publication
(10) Pub. No.: US 2020/0214774 A1
(43) Pub. Date: Jul. 9, 2020

(54) ELECTRIC HANDCART AND SURGICAL ASSIST ROBOT

(71) Applicants: KAWASAKI JUKOGYO KABUSHIKI KAISHA, Kobe-shi (JP); MEDICAROID CORPORATION, Kobe-shi (JP)

(72) Inventors: Toshiaki YOSHIDA, Kobe-shi (JP); Wataru DOI, Kashihara-shi (JP)

(73) Assignees: KAWASAKI JUKOGYO KABUSHIKI KAISHA, Kobe-shi (JP); MEDICAROID CORPORATION, Kobe-shi (JP)

(21) Appl. No.: 16/724,584

(22) Filed: Dec. 23, 2019

(30) Foreign Application Priority Data
Dec. 26, 2018 (JP) 2018-243462

(51) Int. Cl. A61B 34/30; A61B 50/13; B25J 5/00; B25J 13/02; B25J 13/08; B25J 9/16

(52) U.S. Cl. CPC A61B 34/30; A61B 50/13; B25J 5/007; A61B 2034/301; B25J 13/082; B25J 9/1689; B25J 9/1674; B25J 13/025

(57) ABSTRACT

A body including a drive wheel and movable by rotation of the wheel; an electric motor to rotate the drive wheel; a controller to control the electric motor such that the rotational speed of the electric motor is a target rotational speed; an operation input device to receive an input of an amount of operation relating to movement speed of the body; a handle to be used by an operator to maneuver the body, the handle including grips to be grasped by the operator; and a grasping power detection sensor mounted on one of the grips to detect a grasping power with which the operator grasps the one of the grips. The controller determines a gain positively correlated with the grasping power, the amount of the operation as amplified by the gain, and the target rotational speed based on the amount of the operation as amplified by the gain.

### CROSS-REFERENCE TO RELATED APPLICATION

This application claims priority to and the benefit of Japanese Patent Application No. 2018-243462, filed on Dec. 26, 2018, the entire disclosure of which is incorporated herein by reference.

### BACKGROUND OF THE INVENTION

### 1. Field of the Invention

The present invention relates to an electric handcart and a surgical assist robot including the electric handcart.

### 2. Description of the Related Art

Robotic surgery systems for minimally invasive surgery have been conventionally known. A robotic surgery system includes: a surgical assist robot including a surgical instrument manipulator equipped with a surgical instrument and an endoscope manipulator equipped with an endoscope; and a surgeon's console used by a surgeon to remotely control the surgical assist robot. Japanese Laid-Open Patent Application Publication No. 2018-149303 discloses a robot-assisted surgery system of this type.

The robot-assisted surgery system disclosed in Japanese Laid-Open Patent Application Publication No. 2018-149303 includes a surgeon's console and a patient-side cart (surgical assist robot). The patient-side cart includes a mounting base, a support linkage fixedly attached to the mounting base, a platform rotatably coupled to the support linkage, a plurality of set-up linkages fixedly attached to the platform, and surgical instrument manipulators each attached to a corresponding one of the set-up linkages. The mounting base includes a steerable wheel assembly, and this steerable wheel assembly provides for selectively permitting and preventing movement of the mounting base. The mounting base is supported on the floor of a surgery room and can be repositioned into a desired position.

### SUMMARY OF THE INVENTION

In a common robot-assisted surgery system, as described in Japanese Laid-Open Patent Application Publication No. 2018-149303, the base portion of the surgical assist robot (patient-side cart) is configured in the form of a so-called handcart. The operator pushes and pulls the handcart repeatedly to move the surgical assist robot to a target location and then immobilizes the handcart to prevent it from being displaced from the location.

Such a surgical assist robot is heavy and larger than the body of the operator. Thus, it can be envisaged that a handcart equipped with a motor serving to assist an operation force of the operator is used as the base portion of the surgical assist robot.

In general, when a handcart is positioned at a target location, a rough movement operation is first performed in which the handcart is caused to move a relatively long distance toward the target location, and then a fine movement operation is performed in which the handcart is caused to move a relatively short distance in order to precisely position the handcart at the target location. If the level of assistance is adjusted to match the rough movement operation and the same level of assistance is given to the operation force of the operator in both the rough movement operation and the fine movement operation, the operator will have difficulty changing the location of the handcart slightly during the fine movement operation. If the level of assistance is adjusted to match the fine movement operation and the same level of assistance is given to the operation force of the operator in both the rough movement operation and the fine movement operation, the operator will spend a lot of time in the rough movement operation.

The present invention has been made in view of the above circumstances and aims to improve the maneuverability that an electric handcart suitable for use as a base portion of a robot exhibits when positioned at a target location.

An electric handcart according to one aspect of the present invention includes:

a body including a drive wheel and configured to move by rotation of the drive wheel;

an electric motor configured to rotate the drive wheel;

a controller configured to control the electric motor such that the rotational speed of the electric motor is a target rotational speed;

an operation input device configured to receive an input of an amount of operation relating to movement speed of the body;

a handle configured to be used by an operator to maneuver the body, the handle including grips to be grasped by the operator; and

a grasping power detection sensor mounted on one of the grips to detect a grasping power with which the operator grasps the one of the grips, wherein

the controller determines a gain positively correlated with the grasping power, determines the amount of the operation as amplified by the gain, and determines the target rotational speed based on the amount of the operation as amplified by the gain.

In the above electric handcart, the amount of amplification by the gain is large when the operator grasps the grip with great force and small when the operator grasps the grip with small force (or normal force). An increase in the amount of amplification by the gain, namely an increase in the grasping power, leads to an increase in the target rotational speed of the electric motor which is determined as a function of the amount of the operation and hence an increase in the movement speed of the handcart.

For example, when the operator wants to move the electric handcart roughly, the operator grasps the grip with great force. This increases the movement speed achieved by the handcart as a function of the amount of the operation, thereby allowing the handcart to reach a target location quickly. For example, when the operator wants to move the handcart finely, the operator grasps the grip with small force (or normal force). This reduces the movement speed achieved by the handcart as a function of the amount of the operation, thereby making it easy to position the handcart precisely at the target location. Thus, the electric handcart according to the present invention can exhibit improved maneuverability when positioned at a target location.

In the electric handcart, the grips include a rotational grip, the operation input device may include the rotating grip and a rotation sensor configured to detect the rotational position of the rotating grip as the amount of the operation.

In this case, the handle serves both the function of receiving an input of an amount of operation relating to the movement speed and the function of receiving an input of an amplification factor (gain) by which the amount of the operation is amplified. As such, operations to be performed by the operator can be simplified.

A surgical assist robot according to one aspect of the present invention includes at least one manipulator and the above electric handcart supporting the manipulator.

A surgical assist robot according to one aspect of the present invention includes: at least one manipulator including an endoscope or a surgical instrument at a distal end thereof; a positioner supporting the manipulator; and the above electric handcart supporting the positioner.

The electric handcart exhibits improved maneuverability as described above, and is therefore suitable as a base portion of a robot or surgical assist robot capable of both rough movements and fine movements.

The present invention can improve the maneuverability that an electric handcart suitable for use as a base portion of a surgical assist robot exhibits when positioned at a target location.

The above and further objects, features and advantages of the present disclosure will be more apparent from the following detailed description of preferred embodiments with reference to the accompanying drawings.

### BRIEF DESCRIPTION OF THE DRAWINGS

FIG. 1 is a schematic configuration diagram of a surgical assist robot including a handcart according to one embodiment of the present invention.

FIG. 2 is a back view of the handcart.

FIG. 3 is a plan view illustrating the positional relationship among the wheels of the handcart.

FIG. 4 shows the configuration of a power steering mechanism.

FIG. 5 illustrates the configuration of a control system of the handcart.

FIG. 6 shows the internal structure of a handle.

FIG. 7 shows the flow of a process executed by a controller.

FIG. 8 is a graph 1 showing a relationship between a gain and a grasping power (first example).

FIG. 9 is a graph 2 showing a relationship between the gain and the grasping power (second example).

FIG. 10 is a graph 3 showing a relationship between a rotational speed and a grip rotational position as amplified by the gain.

FIG. 11 is a graph 4 showing a relationship between a rotational speed and a grip rotational position.

FIG. 12 shows a modified example of an operation input device.

### DETAILED DESCRIPTION OF THE EMBODIMENTS

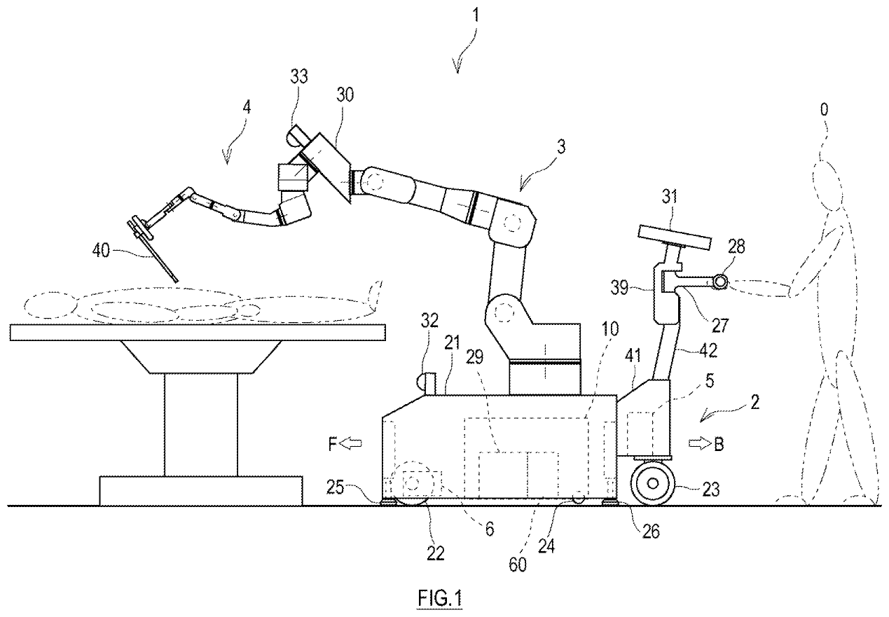

Hereinafter, embodiments of the present invention will be described with reference to the drawings. FIG. 1 is a schematic configuration diagram of a surgical assist robot 1 including an electric handcart according to one embodiment of the present invention (the electric handcart will be simply referred to as “handcart 2” hereinafter). This figure shows an operator O moving the surgical assist robot 1 to the vicinity of a surgery bed on which a patient is placed.

[Overall Configuration of Surgical Assist Robot 1]

The surgical assist robot 1 includes a handcart 2, a positioner 3, a plurality of surgical manipulators 4, and a robot controller 10 in charge of controlling the surgical assist robot 1. The proximal end of the positioner 3 is secured to the handcart 2. The positioner 3 is a so-called manipulator (articulated robot arm), and a platform 30 is mounted on the distal end of the positioner 3. The positioner 3 moves the platform 30 to a desired location and into a desired orientation. The proximal ends of the surgical manipulators 4 are removably coupled to the platform 30. Each of the surgical manipulators 4 is provided at its distal end with an instrument for use in surgery, and this instrument serves as an end effector 40. At least one of the surgical manipulators 4 is an endoscope manipulator equipped at its distal end with an endoscope (robotic endoscope) serving as the end effector 40. At least one of the surgical manipulators 4 is a surgical instrument manipulator equipped at its distal end with a surgical instrument (robotic surgical instrument) serving as the end effector 40.

[Schematic Configuration of Handcart 2]

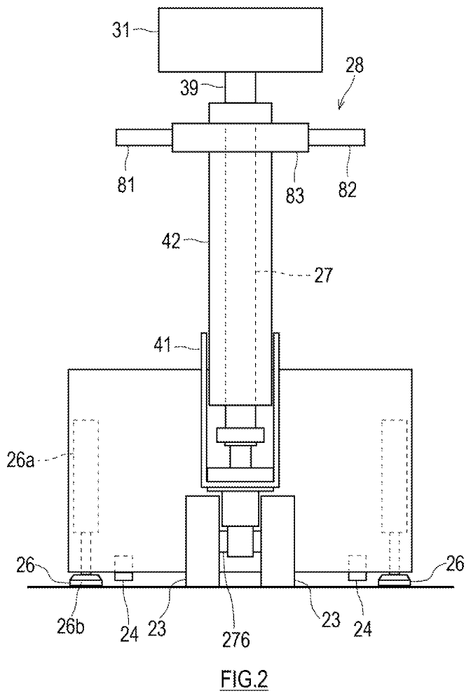

FIG. 2 is a back view of the handcart 2. As shown in FIGS. 1 and 2, the handcart 2 is a so-called electric handcart equipped with an electric motor serving as a drive source. The handcart 2 includes a body 21, drive wheels 22, drive mechanisms 6 for driving the drive wheels 22, steerable wheels 23, a drive mechanism 5 for driving the steerable wheels 23, a handle 28, and a power supply device 29. The power supply device 29 is connected to an external power source and supplies electric power to the drive mechanisms 6 and the drive mechanism 5.

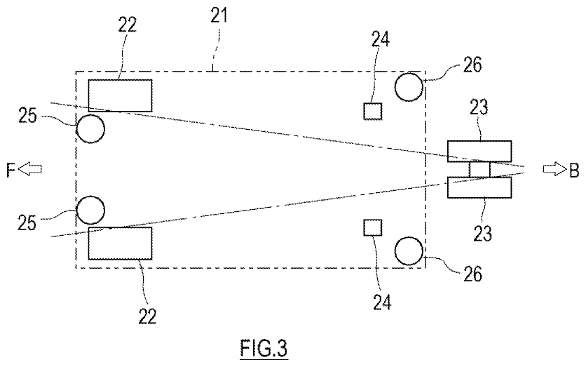

FIG. 3 is a plan view illustrating the positional relationship among the wheels of the handcart 2. As shown in FIGS. 1 and 3, one horizontal direction with respect to the body 21 is defined as a “forward direction F”, and the direction opposite to the forward direction F with respect to the body 21 is defined as a “backward direction B”.

The handcart 2 includes a pair of left and right drive wheels 22 at the front of the body 21 and includes a pair of left and right steerable wheels 23 at the rear of the body 21. The handcart 2 further includes auxiliary wheels 24 disposed between the drive wheels 22 and the steerable wheels 23 in the forward/backward direction. The gauge distance between the steerable wheels 23 is smaller than the gauge distance between the drive wheels 22. Assuming that lines are drawn from the pair of steerable wheels 23 to the left and right drive wheels 22 and a line is drawn between the drive wheels 22, the drawn lines form a triangle in plan view. The auxiliary wheels 24 are located outside the triangle.

A pair of left and right front stabilizers 25 are mounted at the front of the body 21. At the rear of the body 21 are mounted a pair of left and right rear stabilizers 26. As shown in FIG. 2, each rear stabilizer 26 includes a floor-contacting member 26b and an actuator 26a configured to elevate and lower the floor-contacting member 26b. The actuator 26a may be constituted, for example, by an air cylinder and a cylinder rod extendable from and retractable into the air cylinder. The front stabilizers 25 have substantially the same structure as the rear stabilizers 26, and each front stabilizer 25 includes a floor-contacting member 25b and an actuator 25a (see FIG. 5). When the front stabilizers 25 and the rear stabilizers 26 are in contact with the floor, the movement of the handcart 2 is prevented.

The handle 28 is located in the backward direction B relative to the body 21. A steering shaft 27 coupled to the steerable wheels 23 extends upward, and the handle 28 is secured to the upper end of the steering shaft 27. A steering box 41 is coupled to the rear of the body 21, and a steering post 42 enclosing the steering shaft 27 extends upward from the steering box 41. A display device 31 is attached to the steering post 42 via a stay 39.

The display device 31 displays an image (a video) captured by at least one of a first camera 32 mounted on the front of the body 21 and a second camera 33 mounted on the platform 30. The display device 31 is located immediately above and forward of the handle 28 so that the image is easily viewable by the operator O grasping the handle 28. The handle 28 and the display device 31 are located close to each other to allow the operator O to operate the display device 31 with one hand while grasping the handle 28 with the other hand. The operator O grasping the handle 28 can move and steer the handcart 2 while checking the forward environment by means of an image displayed on the display device 31.

FIG. 5 illustrates the configuration of a control system of the handcart. As shown in FIG. 5, the display device 31 includes a display section 31a, an input section 31b, and a control section 31c. The display section 31a may be a touch panel display incorporating the function of the input section 31b. The input section 31b may include at least one operation input element selected from a lever, a button, a dial, a switch, and a touch panel. The control section 31c receives captured image data from at least one of the first camera 32 and the second camera 33, and causes the display section 31a to output the data in the form of a display image. The control section 31c can acquire an operation input received by the input section 31b and change the output of the display section 31a according to the input. The control section 31c is electrically connected to a controller 60 and the robot controller 10. The control section 31c can acquire information from the controller 60 and robot controller 10 and cause the display section 31a to display the information. The control section 31c is configured to acquire an operation input received by the input section 31b and output an operation signal to the controller 60 or robot controller 10 according to the input. For example, the control section 31c can output to the robot controller 10 an operation signal related to the motion of the robot 1. For example, the control section 31c can output to the controller 60 an operation signal related to the motion of the stabilizers 25 and 26. Based on this operation signal, the controller 60 brings the actuators 25a and 26a into operation to elevate or lower the stabilizers 25 and 26.

[Power Steering Mechanism 71]

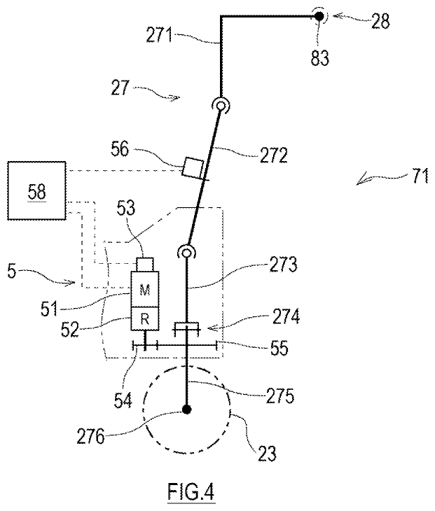

The handcart 2 includes a power steering mechanism 71. The power steering mechanism 71 reduces the load associated with steering of the handle 28. FIG. 4 shows the configuration of the power steering mechanism 71. As shown in FIGS. 2 and 4, the two steerable wheels 23 are coupled by a wheel shaft 276 extending substantially horizontally. To the wheel shaft 276 is coupled a wheel maneuvering shaft 275 extending substantially vertically.

The steering shaft 27 includes an upper operation shaft 271 and a lower operation shaft 273 which are coupled by a universal coupling shaft 272. To the upper end of the upper operation shaft 271 is secured the handle 28. On the universal coupling shaft 272 is mounted a rotation sensor 56 that detects the rotational position of the universal coupling shaft 272. The rotational position detected by the rotation sensor 56 corresponds to the steering angle of the handle 28.

The lower end of the lower operation shaft 273 is connected to the wheel maneuvering shaft 275 via a joint 274. Thus, the rotation of the steering shaft 27 is transmitted to the wheel maneuvering shaft 275. The joint 274 has a play, which provides a dead zone where the rotation of the steering shaft 27 is not transmitted to the wheel maneuvering shaft 275.

Not only the rotation of the steering shaft 27 but also rotational power of the drive mechanism 5 are transmitted to the wheel maneuvering shaft 275. The drive mechanism 5 includes a servo motor 51, a reduction gear 52, a gear 54 fitted around the output shaft of the reduction gear 52, a gear 55 fitted around the wheel maneuvering shaft 275, and a controller 58. The controller 58 causes the servo motor 51 to operate such that the rotational position detected by the rotation sensor 56 and the rotational position of the wheel maneuvering shaft 275 agree with each other. The rotational speed and rotational position of the servo motor 51 are detected by a rotary encoder 53 attached to the servo motor 51, and the detected rotational speed and rotational position are sent to the controller 58 for feedback purposes. As for the rotational output of the servo motor 51, the output torque is amplified by the reduction gear 52 and then transmitted to the wheel maneuvering shaft 275 through the gears 54 and 55. The rotational power thus transmitted to the wheel maneuvering shaft 275 causes rotation of the steering shaft 27 coupled to the wheel maneuvering shaft 275, with the result that the steering operation force applied to the handle 28 is assisted.

[Movement Assist Mechanism 72]

The handcart 2 includes a movement assist mechanism 72. The movement assist mechanism 72 reduces the load associated with pushing and pulling of the handle 28. As shown in FIG. 5, a wheel shaft 65 extending substantially horizontally is coupled to each of the drive wheels 22. The wheel shaft 65 is rotationally driven by the drive mechanism 6. The drive mechanism 6 includes a servo motor 62, a reduction gear 61 that amplifies the output torque of the servo motor 62, a power transmission mechanism 64 that transmits the output of the reduction gear 61 to the wheel shaft 65, and the controller 60 that controls the servo motor 62. The rotational speed and rotational position of the servo motor 62 are detected by a rotary encoder 63 attached to the servo motor 62, and the detected rotational speed and rotational position are sent to the controller 60 for feedback purposes. The drive mechanisms 6 respectively provided for the two drive wheels 22 are substantially the same, and the drive wheels 22 are controlled in a similar manner. Herein, a description is given of one of the drive wheels 22, and a description of the other is omitted.

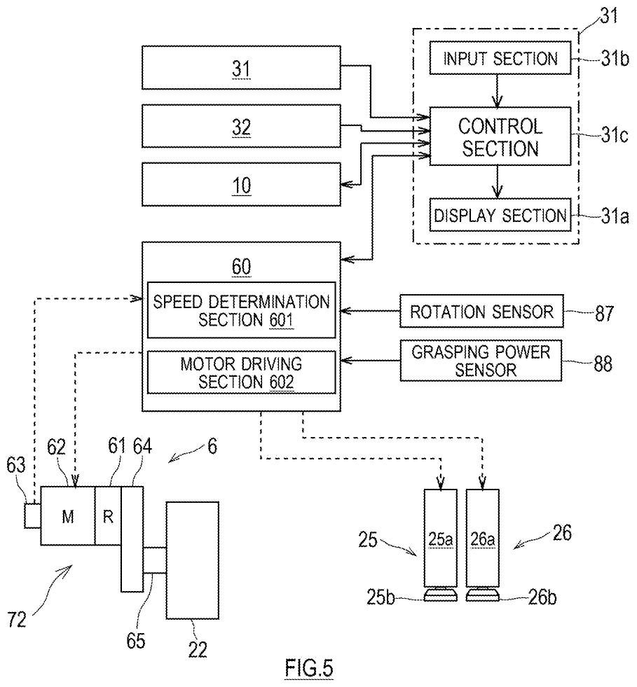

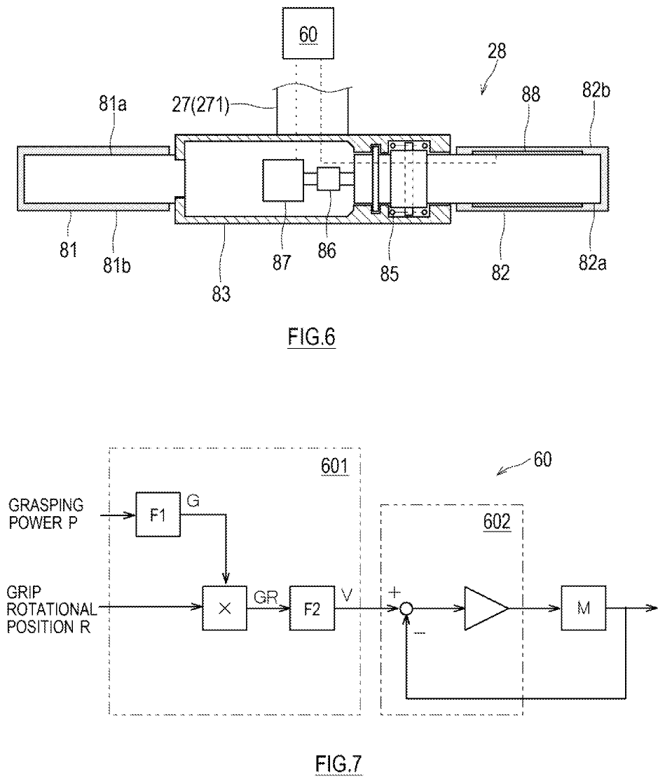

FIG. 6 shows the internal structure of the handle 28. As shown in FIGS. 2 and 6, the handle 28 includes a steering rod 83 secured to the upper end of the steering shaft 27, a stationary grip 81 secured to one of the left and right sides of the steering rod 83, and a rotating grip 82 rotatably supported by the other of the left and right sides of the steering rod 83. The stationary grip 81 includes an inner shaft 81a and a cover 81b enclosing the inner shaft 81a. The rotating grip 82 includes an inner shaft 82a and a cover 82b enclosing the inner shaft 82a. The cover 82b is made of a spongy, soft material.

Between the inner shaft 82a and the cover 82b of the rotating grip 82 is disposed a grasping power detection sensor 88 which is sheet-shaped. The grasping power detection sensor 88 detects the grasping power with which the operator O grasps the rotating grip 82. The grasping power may be detected as a force [N] or a pressure [Pa]. The grasping power detection sensor 88 is electrically connected to the controller 60, and a detection signal of the grasping power detection sensor 88 is output to the controller 60. The grasping power detection sensor 88 according to the present embodiment is a sheet-shaped pressure-sensitive sensor and detects a grasping pressure as the grasping power. The grasping power detection sensor 88 is not limited to this type of sensor, and may be any sensor capable of detecting the power with which the operator O grasps the grip 82 or a variable amount corresponding to the power.

One end of the inner shaft 82a of the rotating grip 82 is inserted into the steering rod 83. The rotating grip 82 is biased by a return spring 85 disposed within the steering rod 83 such that the rotating grip 82 is returnable to a predetermined initial rotational position and such that the load associated with manipulation of the rotating grip increases with increasing amount of rotation from the initial rotational position.

In the steering rod 83, the one end of the inner shaft 82a of the rotating grip 82 and a detection end of a rotation sensor 87 are coupled via a joint 86. The rotation sensor 87 detects the rotational position of the rotating grip 82. The rotation sensor 87 is not limited to a particular one and may be, for example, any sensor such as a potentiometer which is capable of detecting the rotational position (rotational angle) of the rotating grip 82. The rotation sensor 87 is electrically connected to the controller 60, and a detection signal of the rotation sensor 87 is output to the controller 60. At least one of the rotation sensor 87 and the rotating grip 82 has a dead zone such that any operation is not input when the rotational angle of the rotating grip 82 is between an initial rotational angle and a predetermined rotational angle.

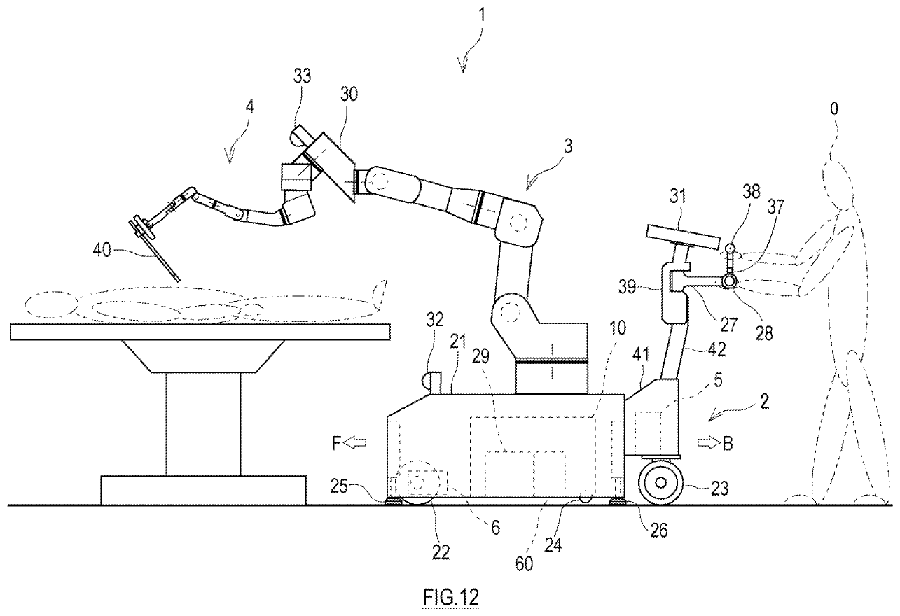

In the present embodiment, the operation input device configured to receive an input of an amount of operation relating to the movement speed of the handcart 2 (body 21) includes the rotating grip 82 and the rotation sensor 87 which detects the rotational position of the rotating grip 82 as the amount of the operation. However, the operation input device is not limited to this configuration. For example, as shown in FIG. 12, a lever 38 mounted on the handle 28 and a sensor 37 configured to detect the displacement of the lever 38 as the amount of the operation (or a rotation sensor configured to detect the rotational displacement of the lever 38 as the amount of the operation) may be used as the operation input device. In this case, at least one of the lever 38 and the sensor 37 configured to detect the displacement of the lever 38 as the amount of the operation may have a dead zone such that any operation is not input when the position of the lever 38 is between an initial position and a predetermined position. As with the case of the rotating grip 82, a dead zone may be provided such that any operation is not input when the rotational angle of the lever 38 is between an initial rotational angle and a predetermined rotational angle. When the rotating grip 82 is used as the operation input device, the width of the dead zone of the rotating grip 82 (the difference between the initial rotational angle and the predetermined rotational angle of the rotating grip 82) may be 0.1 degrees or more and 5 degrees or less or may be 1 degree or more and 5 degrees or less. When the lever 38 configured to be tiltable is used as the operation input device, the width of the dead zone of the lever 38 (the displacement between the initial position and the predetermined position of the lever 38) may be 0.3 mm or more and 15 mm or less or may be 3 mm or more and 15 mm or less. When the lever 38 configured to be rotatable is used as the operation input device, the width of the dead zone of the lever 38 (the difference between the initial rotational angle and the predetermined rotational angle of the lever 38) may be 0.1 degrees or more and 5 degrees or less or may be 1 degree or more and 5 degrees or less.

The controller 60 includes a speed determination section 601 and a motor driving section 602 as functional sections. The speed determination section 601 determines an output rotational speed V of the servo motor 62 and generates a speed command. The speed determination section 601 may be embodied as a computer such as a programmable controller (PLC). The speed determination section 601 includes a processor and volatile and non-volatile storage media (all of which are not shown). The processer is configured as a CPU, an MPU, or a GPU and retrieves and executes various programs stored in the storage media to implement the function of the speed determination section 601. The motor driving section 602 supplies an electric current to the servo motor 62 such that the rotational speed specified by the speed command and the actual rotational speed agree with each other. The motor driving section 602 may be embodied as a servo driver or a servo amplifier.

Hereinafter, how the speed determination section 601 calculates the rotational speed V will be described with reference to FIG. 7. First, the speed determination section 601 uses gain-grasping power information F1 representing a relationship between gain G and grasping power P to determine a value of the gain G corresponding to a value of the grasping power P detected by the grasping power detection sensor 88. The gain-grasping power information F1 is stored in advance in the controller 60. The gain-grasping power information F1 can be freely designed such that the grasping power P and the gain G are positively correlated. The term “positively correlated” means that one of the two variables (i.e., the grasping power P and the gain) increases as the other increases. It should be noted, however, that there may be a zone where one of the two variables remains unchanged (or does not decrease) as the other increases.

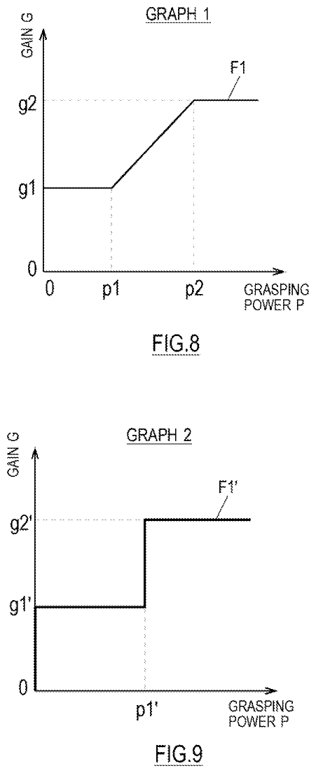

FIG. 8 is a graph 1 showing a relationship between the gain G and the grasping power P as one example (first example) of the gain-grasping power information Fl. In the graph 1, the ordinate represents the gain G, and the abscissa represents the grasping power P. According to the gain-grasping power information F1 shown in the graph 1, when the grasping power P is between 0 and p1, the gain G is constant at g1. When the grasping power P is between p1 and p2, the gain G increases from g1 to g2 with increase in the grasping power P. When the grasping power P is equal to or more than p2, the gain is constant at g2 (0<g1<g2).

FIG. 9 is a graph 2 showing a relationship between the gain G and the grasping power P as another example (second example) of the gain-grasping power information F 1. In the graph 2, the ordinate represents the gain G, and the abscissa represents the grasping power P. According to the gain-grasping power information F1′ shown in graph 2, when the grasping power P is between 0 and p1′, the gain G is constant at g1′. When the grasping power P is equal to or more than p1′, the gain is constant at g2′ (0<g1′<g2′).

Next, the speed determination section 601 multiplies the rotational position R of the rotating grip 82 which is detected by the rotation sensor 87 (i.e., the amount of the operation relating to the movement speed of the handcart 2) by the gain G, and determines a rotational speed V (the target rotational speed of the servo motor 62) corresponding to a rotational position GR as amplified by the gain G. Rotational position-rotational speed information F2 representing a relationship between the rotational position GR of the rotating grip 82 as amplified by the gain and the rotational speed V is stored in advance in the controller 60.

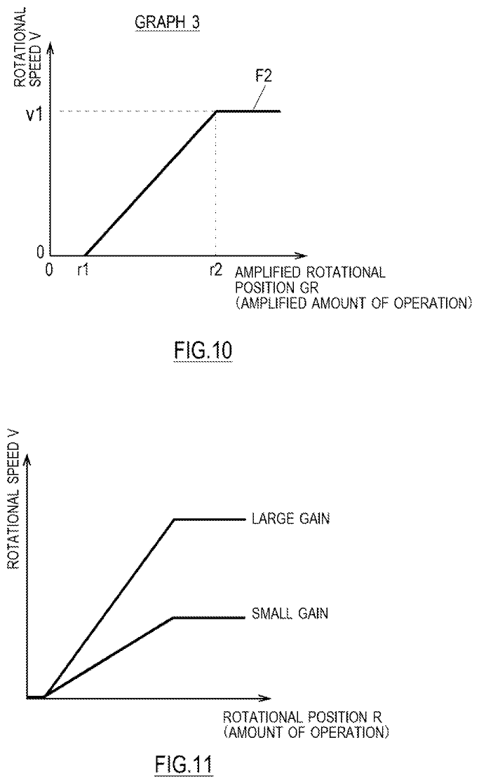

FIG. 10 is a graph 3 showing a relationship between the rotational position GR as amplified by the gain G and the rotational speed V as an example of the rotational position-rotational speed information F2. In the graph 3, the ordinate represents the rotational speed V, and the abscissa represents the rotational position GR as amplified by the gain G. According to the rotational position-rotational speed information F2 shown in graph 3, the zone where the rotational position GR as amplified by the gain G is between 0 and r1 is set as a dead zone, in which the rotational speed V is 0. When the rotational position GR is between r1 and r2, the rotational speed V increases from 0 to v1 with increase in the rotational position GR. When the rotational position GR is equal to or more than r2, the rotational speed V is constant at v1.

Assuming that the above relationship between the rotational position GR as amplified by the gain G and the rotational speed V is converted to a relationship between the rotational position R and the rotational speed V, the relationship resulting from the conversion is as shown in a graph 4 of FIG. 11. A rotational speed V determined for a rotational position R when the gain G is large (i.e., when the grasping power P is high) is higher than a rotational speed V determined for the same rotational position R when the gain G is small (i.e., the grasping power P is low). Thus, if the operator O grasps the rotating grip 82 with great force when rotating the rotating grip 82, the handcart 2 travels fast. If the operator O grasps the rotating grip 82 with small force when rotating the rotating grip 82, the handcart 2 travels slowly.

The movement assist mechanism 72 as described above functions in the following manner in a situation where, for example, the surgical assist robot 1 placed in a corner of a surgery room is moved to a target location in the vicinity of a surgery bed placed at the center of the surgery room.

First, the operator O manipulates the handle 28 while grasping the rotating grip 82 with great force and rotating the rotating grip 82, and thus moves the handcart 2 to nearly the target location. When moving the handcart 2 roughly in this way, the operator O adjusts the gain G to a large value to increase the movement speed of the handcart 2, thereby allowing the handcart 2 to reach the target location quickly.

Next, the operator O manipulates the handle 28 while grasping the rotating grip 82 with small force (or normal force) and rotating the rotating grip 82, and thus precisely position the handcart 2 at the target location. When moving the handcart 2 finely in this way, the operator O reduces the amount of amplification by the gain G; namely, the operator O adjusts the gain G to a small value to reduce the movement speed of the handcart 2. Thus, the operator O can easily perform a slight movement of the handcart 2 to accomplish the positioning of the handcart 2.

As described above, the handcart 2 of the present embodiment includes: a body 21 including drive wheels 22 and configured to move by rotation of the drive wheels 22; a servo motor 62 (electric motor) configured to rotate the drive wheels 22; a controller 60 configured to control the servo motor 62 such that the rotational speed of the servo motor 62 is a target rotational speed; an operation input device (rotating grip 82) configured to receive an input of an amount of operation relating to the movement speed of the body 21; a handle 28 configured to be used by an operator O to maneuver the body 21, the handle 28 including grips 81 and 82 to be grasped by the operator O; and a grasping power detection sensor 88 mounted on the grip 82 to detect a grasping power P with which the operator O grasps the grip 82. The controller 60 determines a gain G positively correlated with the grasping power P, determines the amount of the operation as amplified by the gain G (the rotational position GR), and determines the target rotational speed (rotational speed V) based on the amount of the operation as amplified by the gain (rotational position GR).

In the handcart 2 configured as described above, the amount of amplification by the gain G is large when the operator O grasps the rotating grip 82 with great force and small when the operator O grasps the rotating grip 82 with small force (or normal force). An increase in the amount of amplification by the gain G, namely an increase in the grasping power P, leads to an increase in the movement speed achieved by the handcart 2 as a function of the amount of the operation.

For example, when the operator O wants to move the handcart 2 roughly, the operator O grasps the rotating grip 82 with great force. This increases the movement speed achieved by the handcart 2 as a function of the rotational position of the rotating grip 82 (the amount of the operation relating to the movement speed), thereby allowing the handcart 2 to reach the target location quickly. For example, when the operator O wants to move the handcart 2 finely, the operator O grasps the rotating grip 82 with small force (or normal force). This reduces the movement speed achieved by the handcart 2 as a function of the rotational position of the rotating grip 82 (the amount of the operation relating to the movement speed), thereby making it easy to position the handcart 2 precisely at the target location. Thus, the handcart 2 according to the present embodiment can exhibit improved maneuverability when positioned at a target location.

In the handcart 2 according to the present embodiment, the gain is constant (g2 in FIG. 8 or g1′ in FIG. 9) when the grasping power is in the range of zero to a first predetermined value (p1 in FIG. 8 or p1′ in FIG. 9). As such, the movement speed of the handcart 2 can be prevented from increasing rapidly when the operator O begins to grasp the grip 82.

In the handcart 2 according to the present embodiment, the gain is constant (g2 in FIG. 8 or g2′ in FIG. 9) when the grasping power is equal to or more than a second predetermined value (p2 in FIG. 8 or p1′ in FIG. 9). As such, the movement speed of the handcart 2 can be prevented from increasing unlimitedly.

In the handcart 2 according to the present embodiment, the operation input device includes the rotating grip 82 mounted on the handle 28 and the rotation sensor 87 configured to detect the rotational position of the rotating grip 82 as the amount of operation.

In this handcart 2, the rotating grip 82 of the handle 28 serves both the function of receiving an input of an amount of operation relating to the movement speed of the handcart 2 and the function of receiving an input of an amplification factor (gain) by which the amount of the operation is amplified. This allows the operator O to input two types of operations through one operation element (rotating grip 82). As such, operations to be performed by the operator O can be simplified.

In the handcart 2 according to the present embodiment, the operation input device (rotating grip 82 or lever 38) has a dead zone. In the present embodiment, the operation input device is disposed in the vicinity of the display device 31; thus, when the operator O is operating the display device 31, the operation input device may be accidentally operated as a result of contact of a body part of the operator O with the operation input device. The provision of the dead zone in the operation input device can prevent the movement speed of the handcart 2 from being increased due to an erroneous or unintended operation.

The surgical assist robot 1 according to the embodiment described above includes: at least one surgical manipulator 4 including an endoscope or a surgical instrument at a distal end thereof; a positioner 3 supporting the surgical manipulator 4; and the above handcart 2 supporting the positioner 3. In the surgical assist robot 1 thus configured, the positioner 3 may be omitted, and the surgical manipulator 4 may be supported directly by the handcart 2.

The handcart 2 according to the present embodiment exhibits improved maneuverability as described above, and is therefore suitable as a base portion of a robot (including the surgical assist robot 1) capable of both rough movements and fine movements.

The surgical assist robot 1 according to the embodiment described above further includes a display device 31 including a display section 31a. The handle 28 and the display device 31 are located so close to each other that the operator O can release one hand from the handle 28 and operate the display device 31 with the one hand. As such, the operator O can view the display device 31 to obtain various information while steering the handcart 2.

While a preferred embodiment of the present invention has been described above, the present invention embraces other embodiments provided by modifying the details of the structure and/or functions of the above-described embodiment without departing from the concept of the present invention. Examples of possible modifications to the above-described configuration are as follows.

For example, while in the handcart 2 according to the above embodiment the grasping power detection sensor 88 is mounted on the rotating grip 82, the grasping power detection sensor 88 may be mounted on the stationary grip 81.

While the robot according to the above embodiment is the surgical assist robot 1, the robot according to the present invention is not limited to the surgical assist robot 1. The robot may be any robot including at least one manipulator supported by the handcart 2. The manipulator is not limited to a particular form.

In the power steering mechanism of the handcart 2 according to the above embodiment, the handle 28 and the wheel maneuvering shaft 275 are physically connected via other components, and an operation force applied to the handle 28 is transmitted to the wheel maneuvering shaft 275. The power steering mechanism is not limited to this configuration. For example, in the power steering mechanism as shown in FIG. 4, the lower operation shaft 273 and universal coupling shaft 272 may be omitted, and the rotation sensor 56 may be mounted on the steering shaft 27 coupled to the handle 28. Also in this case, the controller 58 causes the servo motor 51 to operate such that the rotational position detected by the rotation sensor 56 and the rotational position of the wheel maneuvering shaft 275 agree with each other.

1. An electric handcart comprising:
a body comprising a drive wheel and configured to move by rotation of the drive wheel;
an electric motor configured to rotate the drive wheel;
a controller configured to control the electric motor such that the rotational speed of the electric motor is a target rotational speed;
an operation input device configured to receive an input of an amount of operation relating to movement speed of the body;
a handle configured to be used by an operator to maneuver the body, the handle comprising grips to be grasped by the operator; and
a grasping power detection sensor mounted on one of the grips to detect a grasping power with which the operator grasps the one of the grips, wherein
the controller determines a gain positively correlated with the grasping power, determines the amount of the operation as amplified by the gain, and determines the target rotational speed based on the amount of the operation as amplified by the gain.

2. The electric handcart according to claim 1, wherein the gain is constant when the grasping power is in the range of zero to a first predetermined value.

3. The electric handcart according to claim 1, wherein the gain is constant when the grasping power is equal to or more than a second predetermined value.

4. The electric handcart according to claim 1, wherein the grips include a rotating grip, the operation input device comprises the rotating grip and a rotation sensor configured to detect the rotational position of the rotating grip as the amount of the operation.

5. The electric handcart according to claim 1, wherein the operation input device comprises: a lever mounted on the handle; and a sensor configured to detect the displacement of the lever as the amount of the operation or a rotation sensor configured to detect the rotational position of the lever as the amount of the operation.

6. The electric handcart according to claim 4, wherein the grasping power detection sensor is mounted on the rotating grip.

7. The electric handcart according to claim 1, wherein
the grips include a stationary grip,
the operation input device comprises the stationary grip, and
the grasping power detection sensor is mounted on the stationary grip.

8. The electric handcart according to claim 1, wherein the operation input device has a dead zone.

9. The electric handcart according to claim 8, wherein the width of the dead zone is 0.1 degrees or more and 5 degrees or less or 0.3 mm or more and 15 mm or less.

10. A surgical assist robot comprising:
a manipulator comprising an endoscope or a surgical instrument at a distal end thereof; and
an electric handcart supporting the manipulator,
the electric handcart comprising:
a body comprising a drive wheel and configured to move by rotation of the drive wheel;
an electric motor configured to rotate the drive wheel;
a controller configured to control the electric motor such that the rotational speed of the electric motor is a target rotational speed;
an operation input device configured to receive an input of an amount of operation relating to movement speed of the body;
a handle configured to be used by an operator to maneuver the body, the handle comprising grips to be grasped by the operator; and
a grasping power detection sensor mounted on one of the grips to detect a grasping power with which the operator grasps the one of the grips, wherein
the controller determines a gain positively correlated with the grasping power, determines the amount of the operation as amplified by the gain, and determines the target rotational speed based on the amount of the operation as amplified by the gain.

11. The surgical assist robot according to claim 10, wherein the gain is constant when the grasping power is in the range of zero to a first predetermined value.

12. The surgical assist robot according to claim 10, wherein the gain is constant when the grasping power is equal to or more than a second predetermined value.

13. The surgical assist robot according to claim 10, wherein the grips include a rotating grip, the operation input device comprises the rotating grip and a rotation sensor configured to detect the rotational position of the rotating grip as the amount of the operation.

14. The surgical assist robot according to claim 10, wherein the operation input device comprises: a lever mounted on the handle; and a sensor configured to detect the displacement of the lever as the amount of the operation or a rotation sensor configured to detect the rotational position of the lever as the amount of the operation.

15. The surgical assist robot according to claim 13, wherein the grasping power detection sensor is mounted on the rotating grip.

16. The surgical assist robot according to claim 10, wherein the grips include a stationary grip,
the operation input device comprises a stationary grip mounted on the handle, and
the grasping power detection sensor is mounted on the stationary grip.

17. The surgical assist robot according to claim 10, wherein the operation input device has a dead zone.

18. The surgical assist robot according to claim 17, further comprising a display device comprising a display section, wherein the handle and the display device are located close to each other.

19. A surgical assist robot comprising:
at least one manipulator comprising an endoscope or a surgical instrument at a distal end thereof;
a positioner supporting the manipulator; and
an electric handcart supporting the positioner,
the electric handcart comprising:
a body comprising a drive wheel and configured to move by rotation of the drive wheel;
an electric motor configured to rotate the drive wheel;
a controller configured to control the electric motor such that the rotational speed of the electric motor is a target rotational speed;
an operation input device configured to receive an input of an amount of operation relating to movement speed of the body;
a handle configured to be used by an operator to maneuver the body, the handle comprising grips to be grasped by the operator; and
a grasping power detection sensor mounted on one of the grips to detect a grasping power with which the operator grasps the one of the grips, wherein
the controller determines a gain positively correlated with the grasping power, determines the amount of the operation as amplified by the gain, and determines the target rotational speed based on the amount of the operation as amplified by the gain.

20. The surgical assist robot according to claim 19, wherein the gain is constant when the grasping power is in the range of zero to a first predetermined value.
